(19) United States
(12) Patent Application Publication
(10) Pub. No.: US 2025/0155442 A1
(43) Pub. Date: May 15, 2025

(54) APOLIPOPROTEIN A-II BIOMARKERS FOR DETECTING AND MONITORING CANCERS

(71) Applicant: Sun Jet Biotechnology Inc., Taipei City (TW)

(72) Inventor: Yeou-Guang Tsay, Taipei (TW)

(73) Assignee: Sun Jet Biotechnology Inc., Taipei City (TW)

(21) Appl. No.: 18/937,706

(22) Filed: Nov. 5, 2024

Related U.S. Application Data
(60) Provisional application No. 63/597,490, filed on Nov. 9, 2023.

(51) Int. Cl. G01N 33/574; G01N 27/447; G01N 33/92

(52) U.S. Cl. CPC G01N 33/57488; G01N 27/44726; G01N 33/57415; G01N 33/57438; G01N 33/57449; G01N 33/92; G01N 2333/775

(57) ABSTRACT

Disclosed is a method for diagnosing a cancer health state, or a change in cancer health state in a patient, or for diagnosing a risk of the change or presence of a cancer in a patient, comprising determining, in a plasma sample from said patient, one or more biomarker values that corresponds to apolipoprotein A-II (ApoA2)-containing complex structures, and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

### CROSS REFERENCE

This non-provisional application claims the priority under 35 U.S.C. § 119(e) on U.S. Patent Provisional Application No. 63/597,490 filed on Nov. 9, 2023, the entire contents of which are hereby incorporated by reference.

### FIELD OF THE INVENTION

The present invention pertains to a biomarker and a use of biomarker based on apolipoprotein A-II (ApoA2)-containing complex structures for detecting and monitoring cancers.

### BACKGROUND OF THE INVENTION

Hepatocellular carcinoma (HCC) is the most common type of primary liver cancer, accounting for approximately 80% of cases. HCC has a strong male predominance, with a male-to-female ratio estimated to be between 2:1 and 4:1. So far, alpha-fetoprotein (AFP) is the only cancer biomarker widely used in HCC screening. The overall sensitivity of AFP for HCC is approximately 70% for all stages collectively. However, it is not optimal for detecting early-stage HCC with small-sized tumors.

Ovarian cancer (OC) is the third most common gynecologic cancer with poor prognosis and the highest mortality rate. Ovarian cancer is often dubbed a silent killer due to having various symptoms that often only develop when the disease reaches an incurable stage. Currently, CA125 and human epididymis protein 4 (HE4) are the only two markers approved by the FDA for monitoring treatment and detecting disease recurrence for ovarian cancer. While HE4 has a limited role, CA125 has a sensitivity of 55% of stage I and II ovarian cancer.

Breast cancer (BC) is the second most common cancer for women, with approximately 2.5% of these cases resulting in death. Despite a high cure rate for localized disease, only 20% of BC cases are diagnosed at an early stage. International studies have reported that the biomarkers CA15-3 and CA27.29 could be used for BC screening, with sensitivities of 30%-57% and 55%-62%, respectively. For the Taiwanese population, the sensitivity of these two markers is quite low, at 5.5% and 6.4% each.

Overall, there is still a pressing need for biomarkers with better all-around performance in HCC, BC and OC screening. Protein complexes are the bona fide structures that harbor biological functions. In traditional western blotting, these protein complexes are difficult to be accurately quantified due to discrepancy in the electrotransfer of proteins with distinct sizes.

### SUMMARY OF THE INVENTION

The inventors surprisingly found that different species of apolipoprotein A-II (ApoA2) complex structures (or multimeric structures) can be detected and quantified from human plasma sample, and that their levels and ratios are indicative of the risk of cancers including hepatocellular carcinoma (HCC), ovarian cancer (OC), and breast cancer (BC).

In one aspect, the present invention provides a method for diagnosing a cancer health state in a patient, comprising: determining, in a plasma sample from said patient, one or more biomarker values that corresponds to apolipoprotein A-II (ApoA2)-containing complex structures; and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

In one further aspect, the present invention provides a method for diagnosing a change in cancer health state in a patient, comprising: determining, in a plasma sample from said patient, one or more biomarker values that corresponds to apolipoprotein A-II (ApoA2)-containing complex structures; and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

In one yet aspect, the present invention provides a method for diagnosing a risk of the change or presence of a cancer in a patient, comprising: determining, in a plasma sample from said patient, one or more biomarker values that corresponds to apolipoprotein A-II (ApoA2)-containing complex structures; and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

In one further aspect, the present invention provides use of an apolipoprotein A-II (ApoA2)-containing complex structure as a biomarker for a cancer selected from the group consisting of hepatocellular carcinoma (HCC), ovarian cancer (OC), and breast cancer (BC).

In one yet aspect, the present invention provides a capture reagent for apolipoprotein A-II (ApoA2) or an ApoA2-containing complex structure, for use in diagnosing (in vitro) a cancer health state in a patient, wherein said cancer is selected from the group consisting of hepatocellular carcinoma (HCC), ovarian cancer (OC), and breast cancer (BC).

Specifically, the use comprising determining one or more biomarker values that corresponds to ApoA2-containing complex structures using the capture reagent for ApoA2 or ApoA2-containing complex structure.

Also provided is a kit for performing the method as described herein, comprising a capture reagent for ApoA2 or an ApoA2-containing complex structure, and instructions for performing the method.

In some embodiments, the one or more biomarker values are determined by performing an in vitro assay. The in vitro assay may be an immunoassay, including but not limited to a western blotting and a capillary electrophoresis.

In some embodiments, determining the biomarker values comprises performing an in vitro assay, wherein said in vitro assay comprises a capture reagent for ApoA2 or an ApoA2-containing complex structure.

In some embodiments, the capture reagent is an antibody.

In some embodiments, the one or more biomarker values are determined by performing a capillary electrophoresis under non-reducing conditions. According to certain preferred embodiments, the one or more biomarker values include IP₂₀, IP₆₀, IP₁₅₀, or a combination thereof. According to certain preferred embodiments, the assigning is based on a ratio of said biomarker values selected from the group consisting of

$$\frac{{IP}_{60}}{{IP}_{20}} , \frac{{IP}_{150}}{{IP}_{20}} , and \frac{{IP}_{60} + {IP}_{150}}{{IP}_{20}} .$$

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the results of western blotting for analyzing plasma ApoA2 species from three healthy subjects (male) and three hepatocellular carcinoma patients under reducing or non-reducing conditions. The left numbers indicate molecular mass markers (kDa).

FIG. 2 shows the migration profiles of plasma ApoA2 species in capillary electrophoreses for 9 healthy subjects (male), 28 healthy subjects (female), 53 hepatocellular carcinoma patients (male), 9 hepatocellular carcinoma patients (female), 51 ovarian cancer patients, and 161 breast cancer patients. All of the patients have cancers no more severe than stage 2. The left numbers indicate signal percentages relative to the ˜20-kDa peak, while the bottom numbers denote the migration positions of proteins corresponding to their molecular masses. The pair of numbers at the top of the shaded box, that contains P₂₀, P₆₀, or P₁₅₀, indicates the mass range boundaries used for summing signals, such as area Pro representing the ApoA2 dimer. The immunoblot on the right shows how the peaks in capillary western analyses match the structures resolved in SDS-PAGE.

FIG. 3 shows the box plots illustrating the statistical analysis of the ApoA2 indices in patients with hepatocellular carcinoma (male), hepatocellular carcinoma (female), ovarian cancer and breast cancer. The statistical analyses of three ApoA2 multimeric indices were performed. Each dot in the plot represents the index value for an individual subject, with the mean indicated for each group.

FIG. 4 shows the receiver operating characteristic (ROC) curves of the three ApoA2 indices distinguishing between healthy individuals and patients of hepatocellular carcinoma (male), hepatocellular carcinoma (female), ovarian cancer, and breast cancer. Sensitivity is represented on the vertical axis, and (1−specificity) is shown on the horizontal axis for each ApoA2 index cutoff value. Each dot in the plot corresponds to the point with the maximum (sensitivity+specificity). The specific cutoff values are marked by dots, with corresponding performance indicated using dotted lines aligning with the axes.

FIG. 5 shows a summary of the results of two indices for all healthy subjects (HS) and hepatocellular carcinoma (HCC) patients in the present embodiment. Dark grey shading indicates values that are two times higher than the cutoff, whereas light grey shading represents values falling between 1× and 2× the cutoff.

FIG. 6 shows a summary of the results of two ApoA2 indices for all female healthy subjects (HS) and ovarian cancer (OC) patients in this study. Dark grey shading indicates values that are two times higher than the cutoff, whereas light grey shading represents values falling between 1× and 2× the cutoff.

FIG. 7 shows a summary of the results of two ApoA2 indices for all female healthy subjects (HS) and breast cancer (BC) patients in this study. Dark grey shading indicates values that are two times higher than the cutoff, whereas light grey shading represents values falling between 1× and 2× the cutoff.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

The term “biomarker” used herein refers to a measurable characteristic, either within or external to an organism, that indicates a specific physiological state or the presence of a disease. Biomarkers can be used as indicators for assessing physiological processes, disease progression, drug response, or treatment effectiveness. They may include molecules, cells, tissues, physiological indicators, or imaging features, with their changes often closely associated with disease occurrence, progression, treatment response, etc. Biomarkers have significant applications in clinical diagnosis, prediction, monitoring, and treatment, aiding in improving the accuracy of early disease detection, diagnosis, prognosis assessment, as well as evaluating the effectiveness and safety of treatment regimens.

The term “cancer” used herein refers to a group of diseases characterized by the uncontrolled growth and spread of abnormal cells. These cells can invade and destroy surrounding healthy tissues and can also metastasize to distant parts of the body. Cancer can arise from almost any type of cell in the body and may develop in various organs and tissues. It is typically caused by genetic mutations or other factors that disrupt the normal regulation of cell growth and division.

A biomarker value for the biomarkers described herein can be determined using any of a variety of known analytical methods. In some embodiment, the biomarker value can be determined through performing an in vitro assay, for example, an immunoassay. In one embodiment, the determination of a biomarker value involves the use of a capture reagent. A biomarker value may also refer to a ratio calculated based on two or more biomarker values, e.g.,

$$\frac{{IP}_{60}}{{IP}_{20}} , \frac{{IP}_{150}}{{IP}_{20}} , and \frac{{IP}_{60} + {IP}_{150}}{{IP}_{20}} .$$

As used herein, a “capture agent” or “capture reagent” refers to a molecule that is capable of binding specifically to a biomarker. Capture reagents include but are not limited to aptamers, antibodies, antigens, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affibodies, nanobodies, imprinted polymers, avimers, peptidomimetics, a hormone receptor, a cytokine receptor, and synthetic receptors, and modifications and fragments of these.

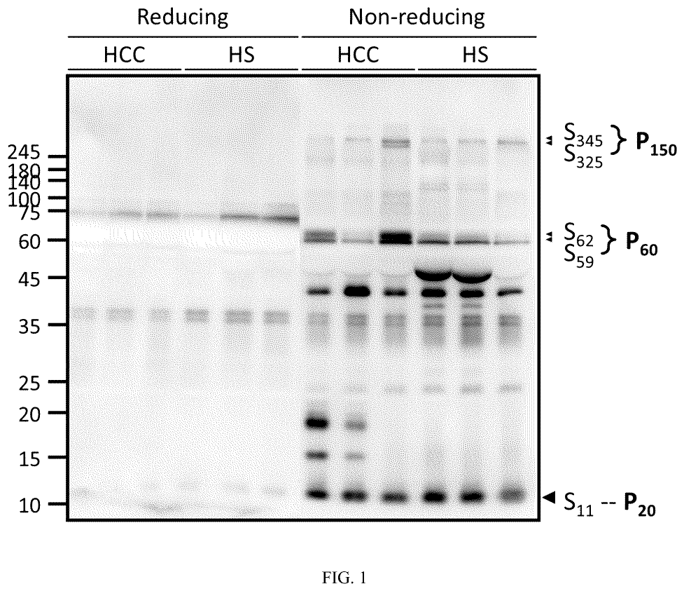

Apolipoprotein A-II (ApoA2), encoded by the APOA2 gene, ranks as the second most abundant among all apolipoproteins found in high-density lipoprotein (HDL) particles. Although there is one report suggesting ApoA2 as a potential serum biomarker for cancer screening, it is important to note that, based on their original data, the distribution of ApoA2 levels in ovarian cancer (OC) patients significantly overlaps with the upper half of those in healthy controls (Timms et al. (2014), Discovery of serum biomarkers of ovarian cancer using complementary proteomic profiling strategies, Proteomics Clin Appl. 8 (11-12): 982-93). Based on these results, finding a reliable criterion for identifying OC patients solely based on ApoA2 levels proves to be a major challenge. ApoA2 has been recognized as monomeric, homodimeric, and ApoA2-ApoD heterodimeric forms in plasma. Notably, the two dimers are supposedly made through disulfide linkages. Recent studies have indicated that the homodimer is the most prevalent form (Kobayashi et al. (2018), Scrum apolipoprotein A2 isoforms in autoimmune pancreatitis, Biochem Biophys Res Commun. 497 (3): 903-907; Wilkins et al. (2021), Spectrum of apolipoprotein AI and apolipoprotein AII proteoforms and their associations with indices of cardiometabolic health: The CARDIA study. J Am Heart Assoc. 2021 Sep. 7;10 (17):e019890), which is largely in line with our findings (see FIG. 1).

The terms “ApoA2 multimer,” “ApoA2-containing complex structure,” and “ApoA2 complex structure” are used interchangeably herein and refer to a protein complex comprising at least one apolipoprotein A-II (ApoA2) subunit, wherein the at least one ApoA2 subunit may be linked to one or more partners (proteins or polypeptides other than ApoA2).

In one aspect, the present invention provides a method for diagnosing a cancer health state, or a change in cancer health state in a patient, or for diagnosing a risk of the change or presence of a cancer in a patient, comprising determining, in a plasma sample from said patient, one or more biomarker values that corresponds to ApoA2-containing complex structures, and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

The one or more biomarker values may include or not include a biomarker value that corresponds to an ApoA2 dimer.

In another aspect, the present invention provides use of an ApoA2-containing complex structure as a biomarker for a cancer selected from the group consisting of HCC, OC, and BC.

The present invention also provides a capture reagent for ApoA2 or an ApoA2-containing complex structure, for use in diagnosing (in vitro) a cancer health state in a patient, wherein said cancer is selected from the group consisting of HCC, OC, and BC. Said use may comprise determining one or more biomarker values that corresponds to ApoA2-containing complex structures using the capture reagent for ApoA2 or ApoA2-containing complex structure.

In one further aspect, the present invention provides a kit for performing the method as described herein, comprising a capture reagent for ApoA2 or an ApoA2-containing complex structure, and instructions for performing the method.

In one further aspect, the present invention provides use of a capture reagent for ApoA2 or an ApoA2-containing complex structure in the preparation of a kit for performing the method as described herein.

According to the present invention, a patient may be assigned as having or not having HCC, OC or BC, or having or not having a change in HCC, OC or BC health state, or having or not having a risk of HCC, OC or BC, based on a higher biomarker value that corresponds to an ApoA2-containing complex structure, or a lower biomarker value that corresponds to an ApoA2-containing complex structure.

As used herein, a higher (biomarker) value or lower (biomarker) value can refer to a value that is higher or lower compared with a reference level. For example, a lower value can be lower than a reference level by at least 30%, 40%, 50%, 60%, 70%, 80%, 90% or 100%; and higher value can be higher than a reference level by at least 30%, 40%, 50%, 60%, 70%, 80%, 90% or 100%. In some embodiments, a reference level can be a standard (or a threshold) value in a normal individual or a control group. For example, a standard or threshold value can be set based on an average or median level obtained from a cohort of normal subjects. In some embodiments, the cohort of subjects can be a population of normal human (without cancer, or without HCC, OC or BC). In addition, a threshold value can be set further based a desired sensitivity and/or specificity for detecting or diagnosing HCC, OC or BC.

According to certain embodiments of the present invention, three species or groups of human plasma ApoA2-containing proteins can be resolved using conventional SDS-PAGE and western blotting under non-reducing conditions, including: (i) an 11-kDa dimeric species, (ii) a 60-kDa group having two protein bands at around of 59 and 62 kDa, and (iii) a 330-kDa group having two protein bands at around 325 and 345 kDa.

According to the present invention, human plasma ApoA2 complex structures can also be resolved by a capillary gel electrophoresis into three major peaks, including P₂₀, P₆₀, and P₁₅₀, and corresponds to the 11-kDa species, the 60-kDa group, and the 330-kDa group, respectively.

A biomarker value is indicative of a concentration of a biomarker, or a ratio of concentrations of the biomarkers in a sample. A biomarker value of the present invention may be a signal intensity or normalized signal intensity of any of the peaks P₂₀, P₆₀, and P₁₅₀, denoted as IP₂₀, IP₆₀, and IP₁₅₀, respectively, or a ratio of the signal intensities. The signal intensity can be measured as the area under the peak. In some embodiments, the signal intensity is measured as the area under the peak in an immunodetection.

In some embodiments, the one or more biomarker values are determined through performing a capillary electrophoresis under non-reducing conditions. Specifically, biomarker signals are detected by performing the capillary electrophoresis and immunodetection, and then the one or more biomarker values are determined based on the detected biomarker signals. According to certain preferred embodiments, the one or more biomarker values include IP₂₀, IP₆₀, IP₁₅₀, or a combination thereof. According to certain preferred embodiments, based on a ratio of said biomarker values selected from the group consisting of

$$\frac{{IP}_{60}}{{IP}_{20}} , \frac{{IP}_{150}}{{IP}_{20}} , and \frac{{IP}_{60} + {IP}_{150}}{{IP}_{20}} ,$$

the patient is assigned as having or not having HCC, OC or BC, or having or not having a change in HCC, OC or BC health state, or having or not having a risk of HCC, OC or BC.

The present invention is further illustrated by the following examples, which are provided for the purpose of demonstration rather than limitation.

### EXAMPLES

### 1. Materials and Methods

### 1.1 Clinical Samples

Pre-operation HCC, BC and OC plasma samples were provided by Dr. Ming-Chih Ho, Dr. Wen-Hong Kuo and Dr. Pao-Ling Torng from the National Taiwan University Hospital. Blood samples were treated with 0.5M EDTA and protease inhibitors before spinning in a benchtop centrifuge at 3000 RPM at 4° C. for 15 minutes. The supernatant was saved as the plasma fraction and stored at −80° C. until usage.

### 1.2 Western Blot Analysis

For each well, 0.3 μL of plasma sample was mixed with SDS-PAGE sample dye, that consists of 0.04 M Tris-HCl pH 6.8, 1 M glycerol, 0.05 M SDS and bromophenol blue, with or without the addition of 0.3 μL of β-mercaptoethanol for reducing or non-reducing analyses, respectively. After incubation at 95° C. for 5 minutes, the sample mixture was loaded into wells in 4% stacking-12% separating Tris-based polyacrylamide gel. After SDS-PAGE, the proteins were transferred to the nitrocellulose membrane in CAPS buffer (0.02 M 3-(cyclohexylamino)-1-propanesulfonic acid in 10% methanol, pH 11). The blot was blocked with 1% BSA in TBST buffer (0.02 M Tris, 0.14 M NaCl, 0.1% Tween 20, pH 7.6), incubated with anti-ApoA2 (Cusabio, CSB-RA001915A0HU, rabbit-derived, diluted 1:2,000) overnight at 4° C. and anti-rabbit HRP conjugate (Jackson, donkey-derived, 711-035-152, diluted 1:10,000) for 1 hour at RT with TBST wash. Horseradish peroxidase-conjugated secondary antibodies were used to give chemiluminescent signals, which were detected in LAS-4000 (Fujifilm, Japan).

### 1.3 Automated Capillary Electrophoresis-Immunodetection

The reagents were purchased from BioTechne, USA, unless specified otherwise. Plasma samples were diluted 1:200, and 5× Fluorescent Master Mix was added to each sample. Incubation took place at 37° C. for 30 minutes in a water bath. Four microliters of each sample were loaded into the top-row wells of plates preloaded with proprietary electrophoresis buffers designed for the separation of proteins ranging from 12 to 230 kDa. The other rows of the plate were filled with 1% bovine serum albumin (Bionovas, AA0530-0250; antibody diluent). The primary and secondary antibody solutions, chemiluminescence reagents, and wash buffer were utilized per the manufacturer's instructions. For the biotinylated SimpleWestern size standard, these rows were filled with antibody diluent and streptavidin-HRP (Genetex, GTX27403) instead of primary and secondary antibody solutions. Anti-ApoA2 (Cusabio, CSB-RA001915A0HU, rabbit-derived, 1:2,000 diluted) was used as primary antibody; anti-rabbit HRP conjugate (Jackson, donkey-derived, 711-035-152, 1:10,000 diluted) was employed as the secondary antibody. The plates were centrifuged for 5 min at 1000 g at room temperature. The plates and capillaries were then loaded into a SimpleWestern™ system (BioTechne USA), and assays were performed using the standard 12- to 230-kDa separation range protocol introduced in version 6.1.0 of the accompanying Compass software. The separation time was set to the default of 25 minutes. Compass software reported data as chemiluminescence signals versus apparent MW. Apparent molecular weights (MW) were determined by aligning size marker peaks with capillary positions and utilizing signals from fluorescently labeled protein standards within the 5× Master Mix to compensate for the variation in migration among capillaries.

### 1.4 Migration Profile Analysis

Analyses of data collected through capillary electrophoresis were carried out with the use of Microsoft Excel 2021 Visual Basic for Applications (VBA). Raw data obtained from the Compass for SW software were converted to text format, and were processed into graphs, with the x-axis representing molecular weight and the y-axis representing the intensity of the immunodetection signals. To identify specific mass ranges, we employed an in-house peak detection and integration program, which was further validated through manual confirmation. These identified ranges were then utilized to sum up the specified peak areas. The sensitivities and specificities for different cutoff values were characterized using receiver operating characteristic (ROC) curve analyses.

### 2. Results

2.1 Circulating ApoA2 Multimeric Species with Sizes of ˜60 kDa and ˜330 kDa Show Significant Increases Compared to the 11-kDa Dimer in HCC Patients

To investigate how significant the changes of ApoA2 multimeric structures were among three patient groups, we conducted western blot analyses following conventional SDS-PAGE under non-reducing conditions. For healthy controls, there were a number of ApoA2-containing species of different sizes, besides the 11-kDa dimeric ApoA2. As most of these species disappeared upon the addition of thiol reagents, they were most likely the conjugated products through disulfide linkage. The analyses of samples from HCC patients showed that most of these ApoA2 species were present, while two groups of protein bands appeared to show significant increase. The ˜60-kDa group contained 59- and 62-kDa species and densitometry showed the signals of 30˜87×10³ AU for healthy subjects and 41˜180×10³ AU for HCC patients. The ˜330-kDa group was made of 325- and 345-kDa bands, whose intensities were 9.5˜15×10³ AU for healthy controls and 3.8˜40×10³ AU for HCC patients (data not shown).

2.2 Peaks P₆₀ and P₁₅₀ Resolved Using Capillary Electrophoresis had Significant Increase with the Dimeric P₂₀ as the Standard in Female Subjects

To quantitatively determine how ApoA2 multimeric structures changed in cancer patients, we attempted to take advantage of an automated system to analyze plasma samples under non-reducing conditions. This platform can resolve ApoA2 multimeric structures into multiple peaks in a migration profile. In the profiles for healthy subjects, there were at least eight peaks clearly discerned, and this complexity is largely consistent with the patterns seen using SDS-PAGE. The dimer is detected as P₂₀ in this system, as it has the highest signals. We had an impression that female subjects appeared to have higher signals in peaks P₆₀ and P₁₅₀. To validate the gender-dependent expression, we employed the same assay to document three indices:

$$\frac{{IP}_{60}}{{IP}_{20}} , \frac{{IP}_{150}}{{IP}_{20}} , and \frac{{IP}_{60} + {IP}_{150}}{{IP}_{20}}$$

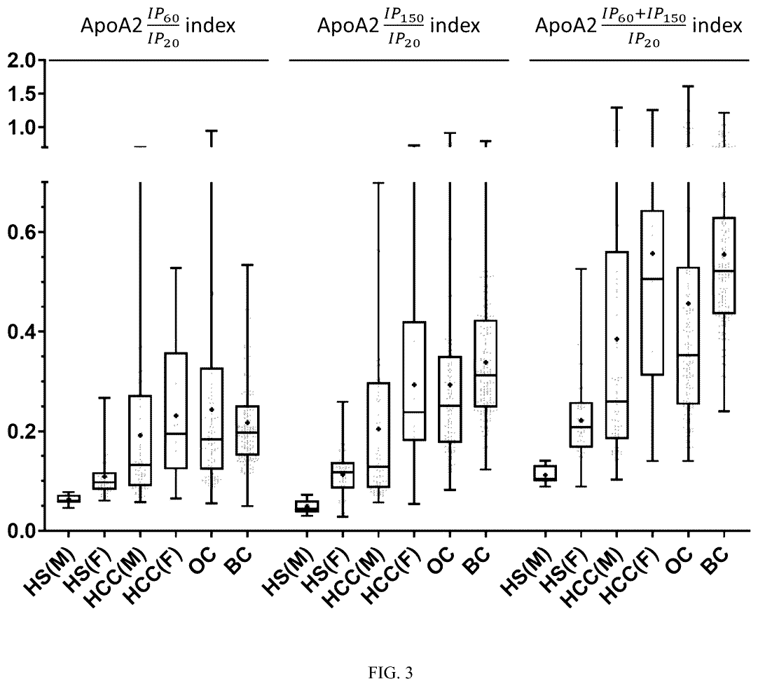

for nine male and 28 female healthy controls. For male subjects, the values were approximately 0.07, 0.04, and 0.12 on average, respectively, while the average values for female controls were 0.09, 0.12, and 0.22 (FIG. 3). All three indices indeed had higher values in female healthy subjects.

Based on the past reports, there is no significant difference in ApoA2 levels between male and female subjects. In a recent report about plasma apolipoprotein profiles, both male and female subjects have ApoA2 concentrations in the range of 25˜50 mg/dl, showing no marked differences. As little is known about disulfide-mediated conjugates of ApoA2, there was no report on our observed sex-dependent change in two ApoA2 conjugates revealed using capillary electrophoresis.

2.3 Peaks P₆₀ and P₁₅₀ Correspond to ˜30 kDa and ˜330 kDa Species Seen with SDS-PAGE have Marked Increases in Three Cancer Patient Groups

For the three patients examined above, their

$$\frac{{P}_{60}}{{P}_{20}} and \frac{{IP}_{150}}{{IP}_{20}}$$

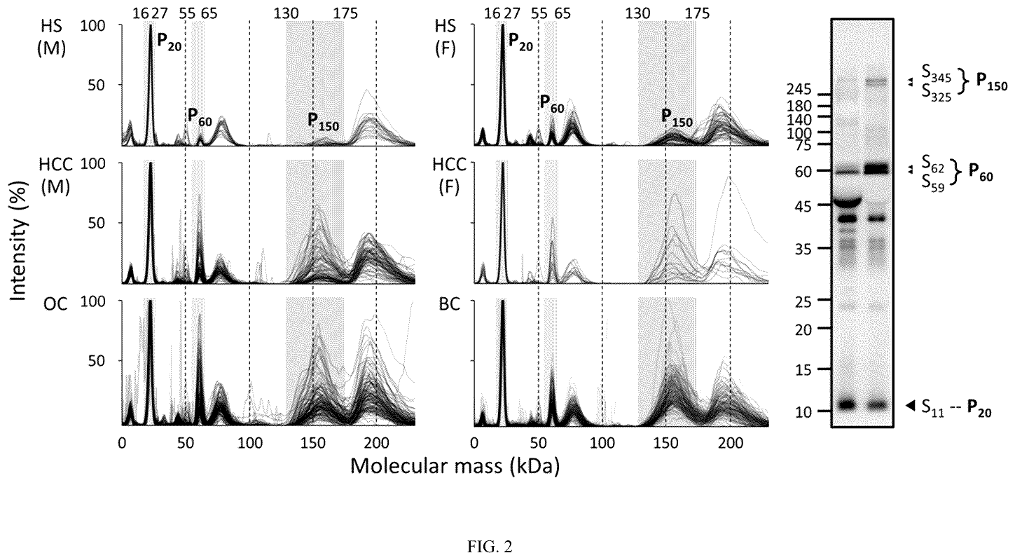

were about 0.06˜0.23 and 0.08˜0.56, while the values for three healthy controls were 0.07˜0.09 and 0.03˜0.06 (data not shown), respectively, which raises a possibility that P₆₀ and P₁₅₀ represent 60 kDa and 330 kDa species which had increases in three HCC patients (FIG. 1). Considering the gender factor, we investigated the increases in two sex groups of stage 1 or 2 HCC patients. For male patients, IP₆₀ and IP₁₅₀ indeed had prominent increases (FIG. 2). The indices

$$\frac{{IP}_{60}}{{IP}_{20}} , \frac{{IP}_{150}}{{IP}_{20}} and \frac{{IP}_{60} + {IP}_{150}}{{IP}_{20}}$$

increased up to 0.19, 0.20 and 0.39 on average in comparison with 0.07, 0.04 and 0.12 for male subjects. While the number of female HCC patients was limited, the three indices of these patients had average values of 0.24, 0.39 and 0.53, much higher than 0.09, 0.12, and 0.21 for female control (FIG. 3). These findings suggest that peaks P₆₀ and P₁₅₀, likely corresponding to 60 kDa and 330 kDa species seen with SDS-PAGE, have marked increases in both male and female HCC patients.

We then used the same analyses to determine the values of three indices in 51 patients with stage 1 to 2 ovarian cancer and 161 patients with stage 0 to 2 breast cancer. In the case of ovarian cancer patients, the multimeric indices

$$\frac{{IP}_{60}}{{IP}_{20}} , \frac{{IP}_{150}}{{IP}_{20}} and \frac{{IP}_{60} + {IP}_{150}}{{IP}_{20}}$$

had means of approximately 0.24, 0.27, and 0.45, respectively (FIG. 3). For breast cancer patients the

$$\frac{{IP}_{60}}{{IP}_{20}} , \frac{{IP}_{150}}{{IP}_{20}} and \frac{{IP}_{60} + {IP}_{150}}{{IP}_{20}}$$

had averaged values of 0.22, 0.33 and 0.55 (FIG. 3). The three indices based on IP₆₀ and IP₁₅₀ have marked increases in three cancer patient groups, and can be used to distinguish these cancer patients from healthy controls.
2.4 all Three ApoA2 Multimeric Indices had Good Performances in Distinguishing HCC, OC, and BC Patients from Healthy Controls
As the quartile analyses revealed good distinction between healthy controls and cancer patient groups (FIG. 3), we decided to use ROC curves to investigate whether it is possible to find cutoffs for these three indices to distinguish patients from healthy controls. Overall, all of these curves are closer to the perfect classifier points than to the random classifier lines. As expected, the cutoff values of these three indices are quite different for two genders. The cutoff values for female subjects were sometimes twice higher than those for males. For different cancers, these indices performed better in identifying male HCC and breast cancer patients.

$$\frac{{IP}_{150}}{{IP}_{20}}$$

appeared to have better performance than

$$\frac{{IP}_{60}}{{IP}_{20}} , and \frac{{IP}_{60} + {IP}_{150}}{{IP}_{20}}$$

can improve the distinguishing power of

$$\frac{{IP}_{150}}{{IP}_{20}}$$

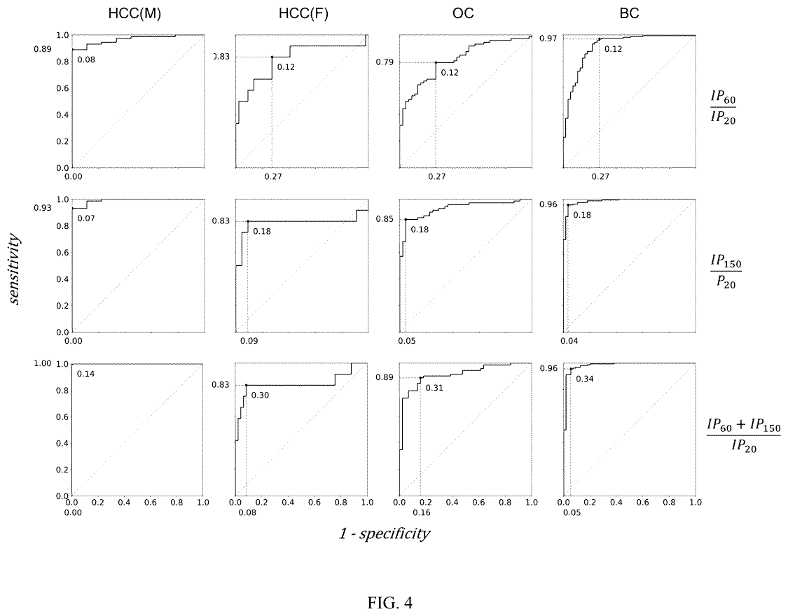

### (FIG. 4).

We then explored the capacity of ApoA2 indices in the screening of cancer patients. Several factors required consideration. First, based on our ROC curve analyses (FIG. 4), we selected IP₁₅₀/IP₂₀ and IP₆₀+IP₁₅₀/IP₂₀ to test how sensitive they were in detection of cancer patients. Second, in clinical practice, a single test can have only one set of criteria. Therefore, the cutoff for each index should be the same for the screening of different cancers. Third, gender effect on ApoA2 multimer expression is quite noticeable. With these considerations, we set the cutoff for

$$ApoA 2 \frac{{IP}_{150}}{{IP}_{20}}$$

at 0.07 for men and 0.18 for women, and that for

$$\frac{{IP}_{60} + {IP}_{150}}{{IP}_{20}}$$

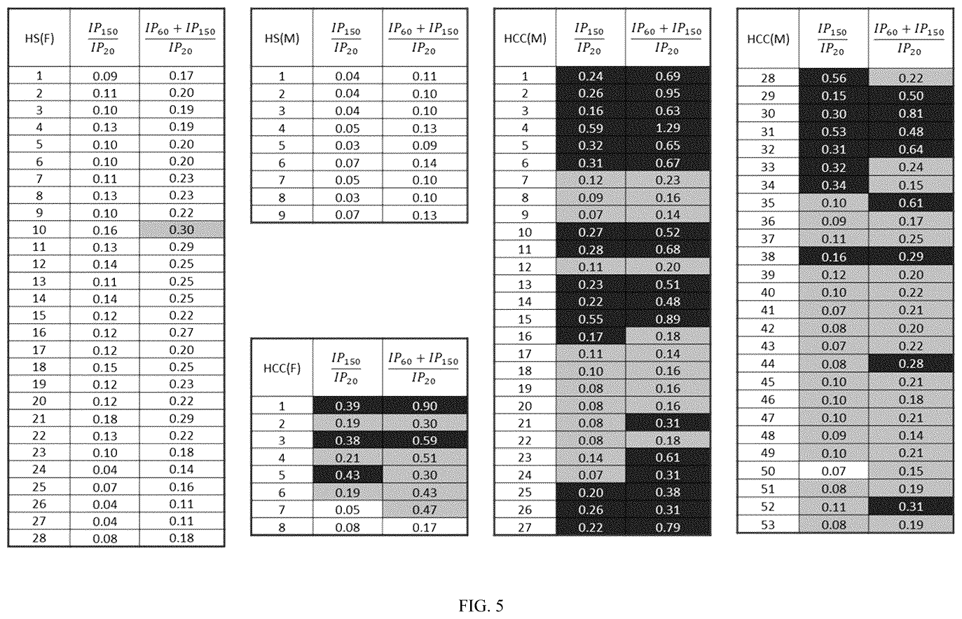

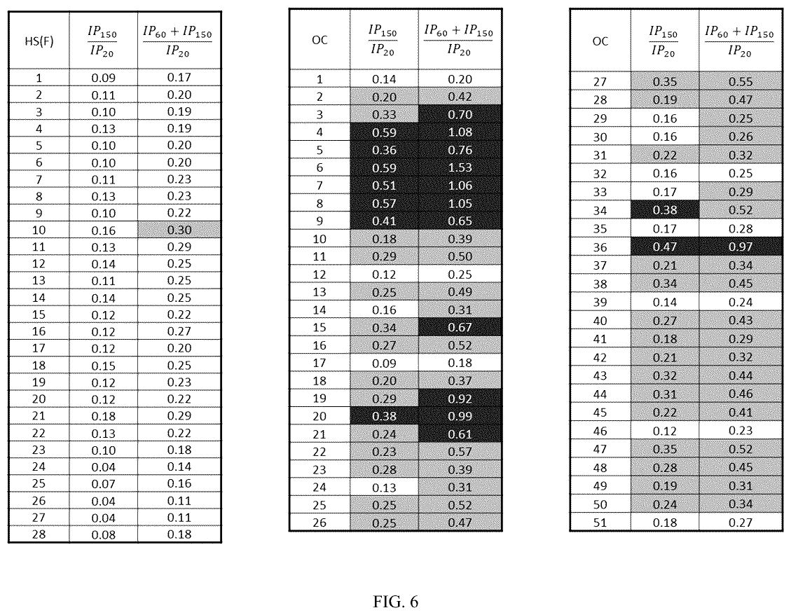

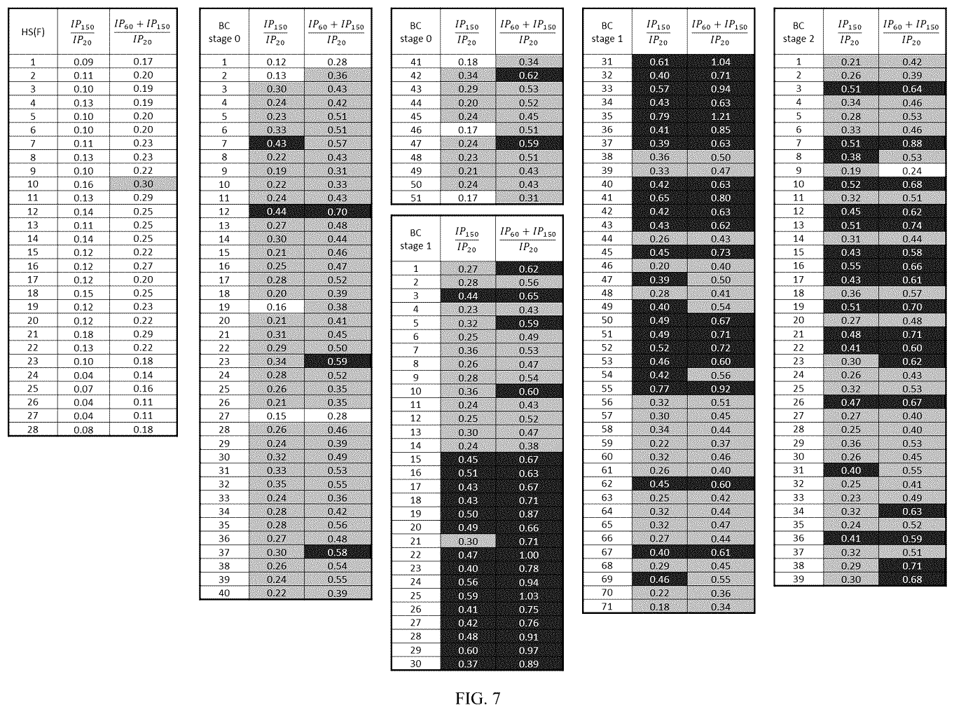

was at 0.14 for male and at 0.29 for female subjects. Using these standards, we could identify 99, 84, and 99% of HCC, OC, and BC patients, respectively (FIGS. 5 to 7). In addition, higher expression was observed for HCC and BC patients, consistent with the results of ROC analyses. For example, more HCC and BC patients had values twice higher than the cutoff than OC patients did (FIGS. 5, 6 and 7). Also, higher expression is indeed associated with later stages. For example, more stage 1 and 2 BC patients had values two times higher than the standard than stage 0 patients (FIG. 7).

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

1. A method for diagnosing a cancer health state in a patient, comprising:
determining, in a plasma sample from said patient, one or more biomarker values that corresponds to apolipoprotein A-II (ApoA2)-containing complex structures; and
assigning the patient as having or not having cancer, or having or not having a change in cancer health state, or having or not having a risk of cancer based on said biomarker values,
wherein said cancer is selected from the group consisting of hepatocellular carcinoma (HCC), ovarian cancer (OC), and breast cancer (BC).

2. The method of claim 1, wherein determining the biomarker values comprises performing an in vitro assay, wherein said in vitro assay comprises a capture reagent for ApoA2 or an ApoA2-containing complex structure.

3. The method of claim 2, wherein the capture reagent is an antibody.

4. The method of claim 2, wherein the in vitro assay is a capillary electrophoresis under non-reducing conditions.

5. The method of claim 2, wherein the one or more biomarker values include IP₂₀, IP₆₀, IP₁₅₀, or a combination thereof.

6. The method of claim 1, wherein the assigning is based on a ratio of said biomarker values selected from the group consisting of
$$\frac{{IP}_{60}}{{IP}_{20}} , \frac{{IP}_{150}}{{IP}_{20}} , and \frac{{IP}_{60} + {IP}_{150}}{{IP}_{20}} .$$

7. A method for diagnosing a risk of the change or presence of a cancer in a patient, comprising:
determining, in a plasma sample from said patient, one or more biomarker values that corresponds to apolipoprotein A-II (ApoA2)-containing complex structures; and
assigning the patient as having or not having cancer, or having or not having a change in cancer health state, or having or not having a risk of cancer based on said biomarker values,
wherein said cancer is selected from the group consisting of hepatocellular carcinoma (HCC), ovarian cancer (OC), and breast cancer (BC).

8. The method of claim 7, wherein determining the biomarker values comprises performing an in vitro assay, wherein said in vitro assay comprises a capture reagent for ApoA2 or an ApoA2-containing complex structure.

9. The method of claim 8, wherein the capture reagent is an antibody.

10. The method of claim 8, wherein the in vitro assay is a capillary electrophoresis under non-reducing conditions.

11. The method of claim 8, wherein the one or more biomarker values based on IP₂₀, IP₆₀, IP₁₅₀, or a combination thereof.

12. The method of claim 7, wherein the assigning is based on a ratio of said biomarker values selected from the group consisting of
$$\frac{{IP}_{60}}{{IP}_{20}} , \frac{{IP}_{150}}{{IP}_{20}} , and \frac{{IP}_{60} + {IP}_{150}}{{IP}_{20}} .$$

13. A kit for performing a method for diagnosing a cancer health state in a patient, comprising a capture reagent for ApoA2 or an ApoA2-containing complex structure, and instructions for performing the method, wherein said cancer is selected from the group consisting of hepatocellular carcinoma (HCC), ovarian cancer (OC), and breast cancer (BC).

14. The kit of claim 13, wherein the method comprising determining one or more biomarker values that corresponds to ApoA2-containing complex structures using the capture reagent, and assigning the patient as having or not having cancer, or having or not having a change in cancer health state, or having or not having a risk of cancer based on said biomarker values.

15. The kit of claim 13, wherein the capture reagent is an antibody.

16. The kit of claim 14, wherein the capture reagent is an antibody.

17. The kit of claim 14, wherein determining the biomarker values comprises performing an in vitro assay using the capture reagent.

18. The kit of claim 17, wherein the in vitro assay is a capillary electrophoresis under non-reducing conditions.

19. The kit of claim 17, wherein the one or more biomarker values include IP₂₀, IP₆₀, IP₁₅₀, or a combination thereof.

20. The kit of claim 14, wherein the assigning is based on a ratio of said biomarker values selected from the group consisting of
$$\frac{{IP}_{60}}{{IP}_{20}} , \frac{{IP}_{150}}{{IP}_{20}} , and \frac{{IP}_{60} + {IP}_{150}}{{IP}_{20}} .$$
